SEQUENCE LISTING (1 sequence)

SEQ ID NO: 1 - DNA, 30 bp, Artificial
  probe DNA sequence
ggggaagtaaggacgcggaaaggatagggg

(19) United States
(12) Patent Application Publication
(10) Pub. No.: US 2005/0084890 A1
(43) Pub. Date: Apr. 21, 2005

(54) High-throughput screening assay for inhibitors of replication protein A

(76) Inventor: John J. Turchi, Beavercreek, OH (US)

Correspondence Address: DINSMORE & SHOHL LLP;One Dayton Centre, Suite 1300, One South Main Street, Dayton, OH, 45402-2023, US

(21) Appl. No.: 10/962,144

(22) Filed: Oct. 7, 2004

Related U.S. Application Data
(60) Provisional application No. 60/509,389, filed on Oct. 7, 2003.

(51) Int. Cl. C12Q 1/68

(52) U.S. Cl. 435/6

(57) ABSTRACT

A homogeneous high-throughput assay is provided which rapidly screens compounds to determine degree of inhibition of Replication Protein A (RPA) binding of DNA. Inhibition by a compounds causes a change in the amount of a detectable signal and the degree of inhibition is determined by comparing the amount of detectable signal with an amount of background detectable signal. The assay comprises adding a volume of RPA to a plurality of wells, adding a volume of each of the plurality of compounds to the plurality of wells, adding reporter-labeled DNA to each of the wells, and measuring the detectable signal emitted from each of the wells. The assay is useful in screening large numbers of compounds for potential utility in cancer treatment strategies, particularly in treatment strategies for chemotherapeutically resistant cancers.

### STATEMENT REGARDING FEDERALLY SPONSORED RESEARCH OR DEVELOPMENT

This invention was made in part using government support under Grant No, CA82741-S1, awarded by the National Institutes of Health The U.S. government has certain rights in this invention.

### FIELD OF THE INVENTION

The present invention relates to replication protein A (RPA) and, more particularly, to a high-throughput screening assay for small-molecule inhibitors of RPA binding to DNA, and to methods which employ the screening assay to modulate disease pathogenesis and/or regulate physiological function.

### BACKGROUND OF THE INVENTION

Replication protein A (RPA) is an attractive target for cancer chemotherapy for numerous reasons. RPA is a heterotrimeric protein consisting of 70, 34, and 14 kDa subunits and is known to bind with high affinity to single-stranded DNA (ssDNA) and with less affinity to double-stranded DNA. RPA is required for the process of chromosomal DNA replication and is essential early in replication during the initiation as well as during the elongation process (Wold M. S., “Replication protein A: A Heterotrimeric, Single-stranded DNA-binding Protein Required for Eukaryotic DNA Metabolism” Annu. Rev. Biochem, 1997; 66: 61-92). RPA is also required for the repair of damaged DNA catalyzed by the nucleotide excision repair (NER) pathway. Id. Further, RPA is required early in the pathway during the damage recognition step. Importantly, RPA has also been very well characterized with respect to its in vivo roles in DNA metabolism, which are mediated largely by its ability to bind single-stranded DNA (ssDNA). In addition, much progress has been made in the structural characterization of RPA. Data obtained from both crystal structures and solution NMR analyses have yielded important information about how RPA achieves its high affinity for binding ssDNA.

RPA's involvement in repair of damaged DNA and replication makes it an attractive target in the search for effective cancer treatment strategies. Interfering with RPA's ability to bind DNA, which ultimately interferes with DNA repair and/or replication, could potentially be useful in developing an effective chemotherapeutic treatment strategy for established cancers. Such a treatment strategy may be particularly beneficial in types of cancer that have become platinum resistant by means of enhanced DNA repair, as, for example, has been observed in recurring, ovarian tumors (Johnson, S. W., Ozals A. F., and Hamilton T. C. “Mechanisms of Drug Resistance in Ovarian Cancer” Cancer, 1993; 71: 644-49).

The domains involved in RPA's DNA binding ability are sensitive to small changes, making these domains possible targets for prevention of the RPA-DNA interaction. This small change may be brought about, for example, by introducing a small molecule into the binding domain of RPA, which could ultimately curb RPA-dependent DNA repair of cisplatin lesions. Extensive characterization of RPA with respect to its DNA binding activity has been conducted, making it a suitable candidate functional relationship around which to develop a high-throughput screen (HTS) capable of monitoring the interaction of RPA and DNA and the effect small molecules have on that interaction. (See Patrick S. M., Turchi, J. J.: “Stopped-flow Kinetic Analysis of Replication Protein A-bindng DNA-damage recognition and Affinity for Single-stranded DNA Reveal Differential Contributions of Rate Constants” J. Biol. Chem., 2001; 276: 22630-37; “Replication Protein A (RPA) Binding to Duplox Cisplatin-damaged DNA is Mediated Through the Generation of Single-Stranded DNA, J. Biol. Chem., 1999; 274: 14972-78; Human Replication Protein A Preferentially Binds Duplox DNA Damaged with Cisplatin”, Biochemistry, 1998; 88014-15; and “Xeroderma Pigmentosum Complementation group A Protein (XPA) modulates RPA-DNA Interactions via Enhanced Complex Stability and Inhibition of Strand Separation Activity, J. Biol. Chem. 2002; 277: 16096-101, all of which are incorporated herein by reference.)

RPA has been shown to be essential for NER of damaged DNA and DNA replication. RPA is a viable target in the treatment of cancer as many chemotherapeutic agents act by blocking DNA replication. Furthermore, inhibition of RPA could prove useful in treating cancers that have acquired resistance to DNA damaging agents through enhanced DNA repair mechanisms, as has been observed for certain platinum resistant carcinomas.

Hence, there is a recognized a need for enhanced methods for identification of inhibitors of RPA.

Large-scale screening approaches to the identification of potential pharmaceutical agents are often expensive and labor-intensive. Thus screening strategies are often limited to those compounds for which some prior basis exists for suspecting likely efficacy. Thus, the range of compounds screened is limited and useful drugs may be overlooked. The ability to screen large numbers of compounds for a specific attribute has been greatly aided by the development of the high-throughput screen (HTS). There are numerous criteria and parameters that must be considered when establishing a HTS. Like in any assay system, the ability to detect the desired activity or phenotype and its modulation by an effector molecule in an efficient manner is essential. Many assay systems for screening compounds that have a particular effect on a target molecular or activity have been modified to be performed in 96-well and more recently in 384-well plates in minimal volumes. The development of accurate detectors for 96-well plate assays has progressed rapidly and sensitive detectors are available for measuring changes in spectral characteristics (UV/Vis), fluorescence, and even radioactivity. These systems allow the simultaneous assaying of larger number of samples in fairly small volumes in a rapid fashion. The sensitivity of these various detectors continues to increase thus making HTSs of this type fairly common. The efficiency of performing HTSs has been facilitated by the use of robotic work stations that can be programmed to perform almost every step in a HTS.

The use of cell based assays to measure cytotoxic activity has resulted in the identification of a large number of active compounds but the specific targets, more often than not, remain unknown. Antibodies have also been utilized extensively in HTSs in an ELISA format and thus allow specific targets to be measured or assayed. These assays and many other HTSs often require numerous washing and incubation steps or filtration steps that extend the total time and hands on time for the assay, as well as increasing the potential for errors and variation within the assay. Minimizing the steps in these assays has been accomplished with fluorescent labeled antibodies, but they still require a number of incubation and washing steps prior to detection. Development of homogenous assays (mix and measure) alleviates many of these problems. Homogenous assays require minimal hands on time and set-up and are becoming increasingly popular in HTSs. In any HTS, specificity remains one of the most important factors in establishing these assays. These efforts have been greatly aided by the use of purified proteins obtained by overexpression in exogenous systems that enable large amounts of active protein to be obtained, thus reducing the cost of performing what can be hundreds of thousands of assays.

Hence, it is an object of the present invention to provide an assay for homogenous high throughput screening of small molecules for RPA inhibition.

It is a further object of the present invention to provide methods which utilize the screening assay to identify molecules useful in strategies directed to modulating physiological functions which involve DNA replication and/or NER.

It is another object of the present invention to provide methods which utilize the screening capability of the HTS assay to develop pharmaceutical agents which may be effective in treatment strategies directed to cancer pathogenesis, and, in particular, to treatment strategies directed to carcinomas which are prone to acquiring resistance to chemotherapeutic treatments.

### SUMMARY OF THE INVENTION

Accordingly, the present invention provides a screening process which is capable of screening a large number of compounds to identify small molecules that have the ability to interact with RPA's DNA binding domains. A homogenous high-throughput screening assay is described herein for measuring RPA's DNA binding activity. Although the present invention is not limited to specific advantages or functionality, it is noted that the assay encompasses a number of the characteristics that are necessary for successful transfer to a HTS. The assay is performed in real time and requires no incubation steps, washing steps or filtration steps and is, therefore, a true homogenous assay.

One embodiment of the present invention provides a homogenous high throughput assay for rapidly screening a plurality of compounds to determine degree of inhibition of RPA binding of DNA by the compounds. The assay comprises: a) adding a volume of RPA to a plurality of wells; b) adding a volume of each of the plurality of compounds to each of the plurality of wells; c) adding a volume of DNA labeled with a reporter to each of the plurality of wells, wherein the reporter emits a detectable signal; d) measuring an amount of detectable signal emitted from each of the plurality of wells; and e) determining the degree of inhibition of RPA binding of DNA in each well by comparing the amount of detectable signal with an amount of background detectable signal wherein the background detectable signal is an amount determined by running the assay without step (b).

Another embodiment provides a homogenous assay for screening a library of compounds for inhibition of RPA binding of DNA. The assay comprises: a) adding a liquid suspension of RPA to a plurality of wells; b) adding a library of compounds to be screened for the inhibition to the plurality of wells; c) adding a fluorescently labeled single-stranded DNA probe to the plurality of wells; d) measuring an amount of fluorescence associated with each well; and determining the degree of the inhibition by one or more of the compounds.

Additional embodiments are directed to methods of treating a condition, disease or disorder comprising: screening compounds according to the assay recited in claim 1; selecting those compounds which show the greatest degree of RPA inhibition; using fluorescence polymerization to exclude those compounds which inhibit RPA via binding with the DNA; formulating a suitable pharmacological composition comprising one or more of any remaining compounds; and administering the suitable pharmacological composition to a subject in need of such treatment. Specific aspects are directed to methods wherein the condition, disease or disorder is treated or benefited by modulating DNA replication, induction of cell apoptosis, inhibiting DNA repair mechanisms along the Nucleotide Excision Repair (NER), and/or modulation of the NER pathway, or some combination thereof.

One particular embodiment provides a screen for a potential therapeutic agent for the enhancement of cancer chemotherapy wherein the enhancement comprises reversal of acquired resistance to DNA damaging agents.

It is contemplated that in some embodiments at least one of the steps of the assay may be performed robotically.

These and other features and advantages of the invention will be more fully understood from the following detailed description of preferred embodiments of the invention taken together with the accompanying figures.

### BRIEF DESCRIPTION OF THE DRAWINGS

The following detailed description of the embodiments of the present invention can be best understood when read in conjunction with the following figures:

FIG. 1 is a plot showing the increase in fluorescence emission of the DNA substrate at varying concentrations of RPA in accordance with one particular aspect of the present invention;

FIG. 2 is a graph showing that the addition of RPA results in a significant and reproducible increase in fluorescence that is an accurate measure of RPA's DNA binding activity in accordance with another particular aspect of the present invention;

FIG. 3 is a plot showing the percent decrease in RPA's DNA binding activity.

FIG. 4 is a plot showing that there is no significant difference in assay sensitivity or stability over a wide range of DMSO concentrations;

FIG. 5 is a plot showing that a number of compounds are capable of inhibiting RPA's DNA binding activity.

### DETAILED DESCRIPTION OF THE PREFERRED EMBODIMENTS

All references cited herein, including patents, patent applications and publications, are hereby incorporated by reference.

DNA replication is a common target for the chemotherapeutic intervention in the treatment of a variety of cancers. This has previously been accomplished by a variety of mechanisms via agents which inhibit DNA synthesis at the level of the extension reaction, a process occurring after initiation of DNA synthesis. The present invention provides methods for identifying compounds which target the processes required for the initiation of DNA replication and which will thus be effective in blocking DNA synthesis, S-phase progression and cell division.

The present invention allows high throughput screening of compounds such as those found in combinatorial libraries to identify compounds which are likely to be efficacious in the inhibition of RPA. The inventive high throughput screening assay is homogenous, meaning that it does not require any separation step (i.e. separation of the bound molecules from the unbound molecules via, e.g. filtration or selective adhesion), allowing for a substantial reduction in time required for such screening and more efficient screening via automation including the employment of robotics technology.

The necessity of RPA and its role in replication of DNA have been clearly established, making it an attractive molecular target for chemotherapeutic intervention in the treatment of cancer. RPA participates in a number of DNA metabolic pathways including DNA replication, NER catalyzed repair of damaged DNA, and homologous recombination. The single-stranded DNA binding activity is likely to be required for each of these pathways in addition to participating in a number of fairly well defined protein-protein interactions.

Without intending to be bound by theory, the high affinity DNA binding OB-folds in the RPA7O subunit are considered the likely targets for small molecule inhibition of RPA's DNA binding activity. The volume within the first OB-fold, amino acids 118-299, is relatively small and a small molecule (mw 100-1000 da) can easily be accommodated. Approximately 3 bases of the ssDNA fit within a single OB-fold. The active site of an OB-fold is populated by both aromatic amino acids and basic amino acids, which could serve to stabilize a small molecule with the correct configuration to allow high affinity binding of a potential inhibitor. Blocking a single OB-fold should be sufficient to dramatically alter the ssDNA binding activity of RPA. Despite the other DNA binding motifs in RPA, the central portion of the RPA7O subunit is thought to be responsible for the initial interaction of RPA with DNA and the correct orientation of DNA within these 2 OB-folds is necessary to then allow the interaction with other DNA binding domains. Likewise, the inability to establish replication forks during S-phase, an expected outcome of RPA inhibition, will likely induce apoptosis (cell suicide) as has been shown in other systems.

RPA has been established as essential for DNA replication both in vivo and in the SV40 in vitro DNA replication system. In SV40 replication, RPA is required early in the DNA replication pathway at the presynthesis stage (Kim, C. and M. S. Wold, 1995, “Recombinant human replication protein A binds to polynucleotides with low cooperativity” Biochemistry 34:2058-2064). RPA is required to stabilize the single-stranded DNA (ssDNA). The ssDNA binding activity is an absolute requirement for efficient replication as mutations in RPA that impair this activity abrogate replication. RPA is also required for the elongation phase of replication, again serving to stabilize single-stranded DNA generated by the DNA helicase leading the fork machinery.

While many of the molecular events required for initiation and the regulation of initiation of replication have been discerned using the SV40 and other lower eukaryotic model systems, RPA has been found to play an important role in this process in higher eukaryotic systems as well. The finding that RPA participates during the initiation process is not surprising. The timing of RPA association with active origins just prior to initiation suggests that this is an excellent position for intervention in attempts to disrupt the initiation of DNA replication. The cumulative data obtained from the various systems, SV-40, yeast and higher eukaryotes, establishes RPA as an essential component of the molecular machinery required for initiation of DNA replication.

RPA is well-known to play an essential role in NER catalyzed repair of damaged DNA and has been demonstrated to play important roles in base excision repair (BER) and repair of interstrand crosslinks, as monoclonal antibodies directed against RPA inhibit repair synthesis on these damaged DNA molecules (DeMott, M., S. Zigman, and R. Bambara. 1998. “Replication protein A stimulates long patch DNA base excision repair” J. Biol. Chem. 273:27492-27498 and Dianov, G., B. Jensen, M. Kenny, and V. Bohr. 1999. “Replication protein A stimulates proliferating cell nuclear antigen-dependent repair of basic sites in DNA by human cell extracts” Biochemistry 38:11021-11025).

In the NER pathway, RPA is involved at an early step in the pathway—the recognition of DNA damage. NER is known to be responsible for the repair of a variety of bulky DNA lesions. NER can be divided into 5 steps including recognition, incision, excision, resynthesis and ligation. The recognition process is thought to depend on two parameters of the damaged DNA, the first is a structural distortion in the DNA duplex induced by the DNA adduct and the second is the actual chemical bass modification. Work performed in the laboratory of the present inventor suggests that RPA is responsible, though likely not sufficient for the first aspect of damage recognition, i.e., the helix destabilizing aspect of DNA damage recognition (Patrick and Turchi, 1998, supra; Patrick and Turchi, 1999, supra). It was demonstrated that RPA binds to duplex cisplatin-damaged DNA by recognizing the distortion of the duplex and not the actual chemical modification of the DNA (Patrick and Turchi, 1999, supra).

RPA also participates in some protein-protein interactions which are essential to the NER pathway, independent of RPA's DNA binding activity. XPA is a 31 kDa protein with a Zn finger DNA binding motif that is also required for NER (Aboussekhra, A., Biggerstaff, M., Shivji, M. K., Vilpo, J. A., Moncollin, V., Podust, V. N., Protic, M., Hubscher, U., Egly, J. M., and Wood, R. D. (1995) Cell 80, 859-868). RPA also participates in protein-protein interactions with each of the excision repair nucleases, XPG and ERCC1/XPF (Matsunaga, T., Park, C. H., Bessho, T., Mu, D., and Sancar, A. (1996) J. Biol. Chem. 271, 11047-11050). These interactions position the two nucleases upstream and downstream of the lesion to allow incision of the damaged strand 5-8 bases 3′ and 22-25 bases 5′ of the damage (Park, M. S., Ludwig, D. L., Stigger, E., and Lee, S.-H. (1996) J. Biol. Chem. 271, 18996-19000). Mutations in RPA that disrupt either the DNA binding activity or RPA's ability to interact with any of these proteins result in a defect in NER catalyzed repair of damaged DNA.

The removal of damaged DNA by means such as NER is essential for cell survival, and the ability to inhibit this removal would be expected to result in a more effective cancer treatment. RPA's early involvement in the NER pathway makes it a viable target.

DNA alkylating agents are commonly used to treat a variety of cancers. The removal of the resultant “desirable” DNA lesions by repair processes is then detrimental in the context of cancer treatment. The ability to inhibit the repair of these lesions would, therefore, be expected to result in a more effective treatment. This is borne out in vivo as XP individuals, who all have mutations in genes encoding proteins involved in the NER process, are hypersensitive to DNA damaging agents including cisplatin (Cleaver, J. 1971. “Repair of alkylation damage in ultraviolet-sensitive (xeroderma pigmentosum) human cells” Mutat. Res. 12:453-462).

Tumor resistance to alkylating agents has been at least partially attributed to increased DNA repair capabilities (Hamilton et al. “Mechanisms of resistance to cisplatin and alkylating agents,” Cancer Treatment Research, 1989; 4B: 151-160). Therefore, identifying agents that inhibit the NER process could be expected to synergize with traditional DNA damaging agents, and also be effective in certain drug resistant cancers. Cisplatin (cis-Diaminedichloroplatinum(II)) is a widely prescribed chemotherapeutic agent used in the treatment of cancer. The intracellular target of cisplatin is DNA, and cisplatin-DNA adducts are thought to impart its chemotherapeutic efficacy. Resistance to cisplatin is a common clinical problem and cellular repair of cisplatin-DNA adducts has been suggested as one mechanism of cisplatin resistance (Perez, R. P., Hamilton, T. C., Ozols, R. F., Young, R. C. (1993) Cancer 71, 1571-1580).

Targeted therapies in treating cancer are becoming more popular as advances in our understanding of the pathways and processes in cancer cell development and progression continue. In the pathogenesis of some cancers, however, acquired resistance to treatment is a serious issue and a high incidence of cancer recurrence results among cancers particularly prone to acquired resistance. For example, despite many of the advances in the treatment of ovarian cancer, approximately 14,000 women will die of the disease this year and nearly 23,000 new cases will be diagnosed. Of the women diagnosed, only 25% are identified early enough to give them a 95% chance of surviving 5 years and women diagnosed in later stages have only a 50% change of surviving 5 years. Of the women that do survive, nearly 50% will have multiple recurrences. Clearly, there is a need for better treatment options for those diagnosed with resistant ovarian cancer.

Recurrent cancer is often associated with acquired resistance, which is usually multifactorial in nature. Acquired resistance to DNA damaging agents, such as cisplatin, through enhanced DNA repair has been observed for certain carcinomas (Hamilton et al. “Mechanisms of resistance to cisplatin and alkylating agents,” Cancer Treatment Research, 1989; 4B: 151-160). One mechanism for increasing the sensitivity of resistant cancers to cisplatin is the inhibition of the repair systems responsible for removal of cisplatin DNA lesions. RPA is required for this process. The present invention is directed to methods for identifying and developing the small molecule inhibitors that will block or disrupt the interaction of RPA with DNA, therefore blocking repair of DNA lesions.

In accordance with one embodiment of the present invention, a homogenous high throughput assay for rapidly screening a plurality of compounds to determine degree of inhibition of RPA binding of DNA is provided. The assay comprises: a) adding a volume of RPA to a plurality of wells; b) adding a volume of each of the plurality of compounds to each of the plurality of wells; c) adding a volume of DNA labeled with a reporter to each of the plurality of wells, wherein the reporter emits a detectable signal; d) measuring an amount of detectable signal emitted from each of the plurality of wells; and e) determining the degree of inhibition of RPA binding of DNA in each well by comparing the amount of detectable signal with an amount of background detectable signal wherein the background detectable signal is an amount determined by running the assay without step (b). In a further embodiment of the assay at least one of the steps is performed robotically.

Specifically, the percentage inhibition is calculated by taking the difference between the fluorescence values of reactions containing DNA and RPA and reactions containing DNA, RPA, and the test compound and dividing by the difference between the fluorescence values of reactions with DNA and RPA and the fluorescence of the DNA alone. This value is then multiplied by 100 to give percentage inhibition. The measurement is typically reported as fluorescence above the background fluorescence of the reporter alone. In one aspect the of the assay the DNA is single stranded DNA.

In another embodiment, the assay further comprises a secondary screening step to identify the mechanism of inhibition. The secondary screening step comprises fluorescence polymerization. Fluorescence polarization allows a more precise determination of whether the method of inhibition is via an interaction with DNA or RPA. Fluorescence polarization is a measure of the rate at which an object tumbles or rotates in three-dimensional space. A flurorescent labeled DNA is employed which results in a baseline polarization that is increased upon RPA binding; to the DNA, slowing its rotation. It can then be determined if the interaction of the compound was with DNA or RPA by constructing binding isotherms while varying RPA at a fixed concentration of DNA with and without the addition of specific compounds. Using fluorescence polarization, compounds that exert their inhibitory effect through an interaction with the DNA are capable of being identified.

In one specific aspect of the assay the reporter is a fluorescent reporter and the signal is fluorescence emission. In a more specific aspect, the reporter comprises 5′ purine-bound fluorescein. In a very specific aspect the single stranded DNA comprises 5′ purine-bound fluorescein which shows an increase in fluorescence that is directly proportional to its binding with RPA, and in an even more specific aspect, the single stranded DNA comprises SEQ ID NO: 1. In a particular embodiment of the assay, the plurality of compounds is a library of compounds.

A further embodiment of the invention provides a fluorescently labeled single stranded DNA probe consisting essentially of SEQ ID NO: 1 bound with a fluorescent label. In this embodiment the fluorescent reporter consists of a 30 base DNA containing a 5′ fluorescein label. The 30 base reporter DNA was purified and the sequence of the probe DNA is

(SEQ ID NO: 1)

5′-GGGGAAGTAAGGACGCGGAAAGGATAGGGG-3′.

An embodiment directed to a homogenous assay for screening a library of compounds for inhibition of RPA binding of DNA is also provided. The assay comprises: a) adding a liquid suspension of RPA to a plurality of wells; b) adding a library of compounds to be screened for the inhibition to the plurality of wells; c) adding a fluorescently labeled single-stranded DNA probe to the plurality of wells; d) measuring an amount of fluorescence associated with each well; and e) determining the degree of the inhibition by one or more of the compounds. In one specific aspect of this assay the fluorescently labeled single-stranded DNA probe comprises 5′ purine-bound fluorescein and in a very specific aspect the single stranded DNA comprises SEQ ID NO: 1.

Hence, in accordance with another embodiment of the invention, a method is provided of treating a condition, disease or disorder by modulating DNA replication. The method comprises: a) screening compounds according to the inventive homogeneous high-throughput assay; b) selecting those compounds which show the greatest degree of RPA inhibition; c) using fluorescence polymerization to exclude those compounds which inhibit RPA via binding with the DNA; d) formulating a pharmacological composition comprising one or more of any remaining compounds; and e) administering the pharmacological composition to a subject in need of such treatment. In a particular embodiment of this method the condition, disease or disorder is cancer and in a specific aspect the condition, disease or disorder is acquired resistance to chemotherapeutic agents. In an even more specific aspect the condition, disease or disorder is a platinum-resistant carcinoma, and in accordance with a very specific aspect the condition, disease or disorder is ovarian cancer.

In accordance with a further embodiment of the invention, a method of treating a condition, disease or disorder benefited by the induction of cell apoptosis (cell-suicide) is provided. The method comprises: a) screening compounds according to one embodiment of the inventive homogeneous high-throughput screening assay; b) selecting those compounds which show the greatest degree of RPA inhibition; c) using fluorescence polymerization to exclude those compounds which inhibit RPA via binding with the DNA; d) formulating a pharmacological composition comprising one or more of any remaining compounds; and e) administering the pharmacological composition to a subject in need of such treatment. In one aspect of the inventive method the condition, disease or disorder is cancer. In a more specific aspect the condition, disease or disorder is acquired resistance to chemotherapeutic agents. In a very specific aspect the condition, disease or disorder is a platinum-resistant carcinoma, and in an even more specific aspect the condition, disease or disorder is ovarian cancer.

A further embodiment of the invention is directed to a method for inhibiting DNA repair mechanisms along the Nucleotide Excision Repair (NER) pathway. The method comprises: screening compounds according to an embodiment of the inventive homogeneous high-throughput screening assay; selecting those compounds which show the greatest degree of RPA inhibition; using fluorescence polymerization to exclude those compounds which inhibit RPA via binding with the DNA; formulating a pharmacological composition comprising one or more of any remaining compounds; and administering the pharmacological composition to a subject in need of such treatment.

In accordance with another embodiment of the invention, a method of treating a condition, disease or disorder by modulation of the NER pathway is provided. The method comprises: a) screening compounds according to the assay recited in claim 1; b) selecting those compounds which show the greatest degree of RPA inhibition; c) using fluorescence polymerization to exclude those compounds which inhibit RPA via binding with the DNA; d) formulating a suitable pharmacological composition comprising one or more of any remaining compounds; and e) administering the suitable pharmacological composition to a subject in need of such treatment. In one aspect of the method the condition, disease or disorder is cancer, and in a specific aspect the condition, disease or disorder is acquired resistance to chemotherapeutic agents. In a very specific aspect the condition, disease or disorder is a platinum-resistant carcinoma and in an even more specific aspect the condition, disease or disorder is ovarian cancer.

One embodiment provides a screen for a potential therapeutic agent for the enhancement of cancer chemotherapy. The enhancement comprises reversal of acquired resistance to DNA damaging agents, and the screen comprises an embodiment of the inventive homogeneous high-throughput assay described prior.

The invention is illustrated by the following example, which is not intended to limit the scope thereof.

### EXAMPLE

In accordance with one exemplary embodiment of the present invention, a homogeneous high-throughput screening assay is provided that employs a novel fluorescent reporter for measuring RPA's DNA binding activity. The assay can be performed in 96-well plates, wherein compounds to be screened are added to RPA and then the fluorescent probe for DNA binding is added. The fluorescence is then quantified in a 96-well plate fluorescence spectrophotometer.

Using this assay, a collection of small molecules were screened and the effect of these molecules on the DNA binding activity of RPA was assessed. Of the 2000 compounds screened, 79 scored positive for inhibition of RPA binding activity. Secondary screening of these compounds was performed using an electrophoretic mobility shift assay (EMSA), and of the 79 compounds 9 scored positive. These 9 were characterized further in titration experiments to determine the most potent inhibitor and resulted in a number of compounds showing an IC₅₀ in the low micro molar range. Fluorescence polarization analyses were also performed to determine the mechanism of inhibition for each compound. Validation of the inhibitory activity of selected compounds was verified using in vitro NER catalyzed excision of a single cisplatin-lesion in a duplex DNA. These results demonstrate the utility of high-throughput assays for identifying inhibitors of RPA. The identification of inhibitors of RPA and it's activity in NER will be useful in the characterization of RPA and its interaction with DNA and also may allow modulation of NER activity to circumvent resistance to certain chemotherapeutic agents.

Materials and Methods.

Fluorescent Labeled DNA Substrate.

The fluorescent reporter consists of a 30 base DNA containing a 5′ fluorescein label. The DNA oligonucleotides were purchased from Integrated DNA Technologies, Inc. (Coralville, Iowa). The 30 base reporter DNA was purified by electrophoresis on a 15% polyacrylamide/7 M urea preparation DNA sequencing gel. The sequence of the probe DNA is.

5′-GGGGAAGTAAGGACGCGGAAAGGATAGGGG-3′.

Protein Purification.

Recombinant human RPA was overexpressed in Escherichia coli using the vector pdT11 provided by Marc Wold. RPA expression was induced by the addition of IPTG to 0˜4 mM and protein purified as described in Henricksen, L. A., and Wold, M. S. (1994) J. Biol. Chem. 269, 24203-24208. Approximately 5 mg of purified protein was obtained from a 2 L culture.

High Through-Put Assay.

RPA (10 pmol) was added to each well of a 96-well plate in a volume of 100 μl. Individual compounds were added to each well followed by the fluorescent reporter. The final reaction volume was 200 μl. Plates were read in a fluorometer. Fluorescence emission was read at 518 nm following excitation at 498 mm. Fluorescence was read using a 96-well plate reader with a 2.5 nm excitation slit width and a 5 nm emission slit width. To calculate the percentage inhibition, the difference between the fluorescence values of reactions containing DNA and RPA and reactions containing DNA, RPA, and reactions containing DNA, RPA, and the test compound was divided by the difference between the fluorescence values of reactions with DNA and RPA and the fluorescence of the DNA alone. This value was then multiplied by 100 to give percentage inhibition. The data is presented as fluorescence above the background fluorescence of the reporter alone unless indicated otherwise.

Development of HTS

In an effort to investigate the effect small molecules have on the interaction of RPA with DNA, an HTS assay was developed that relies on the use of a fluorescein-labeled ssDNA substrate. This purine-rich DNA substrate is 5′ fluorescein-labeled and shows an increase in fluorescence that is proportional to its binding with RPA. When RPA was titrated into a reaction containing the fluorescein-labeled DNA, a plot of fluorescence versus RPA concentration reveals a hyperbolic curve that saturated at a 1:1 ratio of RPA to DNA. In an effort to optimize the detection, fluorescent excitation and emission wavelengths were tested along with varying slit widths and voltages. The optimal parameters were excitation at 498 nm and emission at 518 mm. The optimal excitation and emission slit widths and voltage were 2.5 and 5 nm and 800 V, respectively.

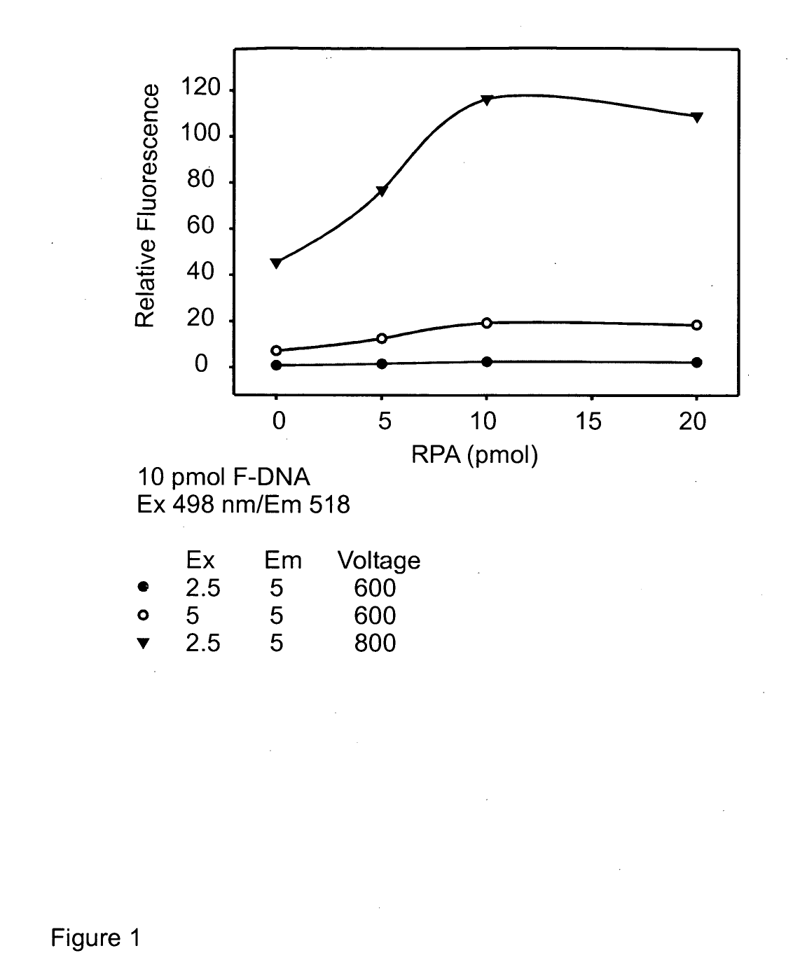

In an attempt to test a large number of compounds simultaneously, the assay was converted to a 96-well plate format. In this assay, the fluorescein-labeled DNA was diluted into assay buffer and 100 μl were dispensed into each well. The fluorescence of each well was then read. Each well then received 100 μl RPA and the fluorescence of each well was read again. Percentage increase of fluorescence was then calculated from these two readings and is represented in FIG. 1. This figure illustrates a significant increase in fluorescence as DNA and RPA bind, as well as demonstrates reproducibility across the 96-well plate.

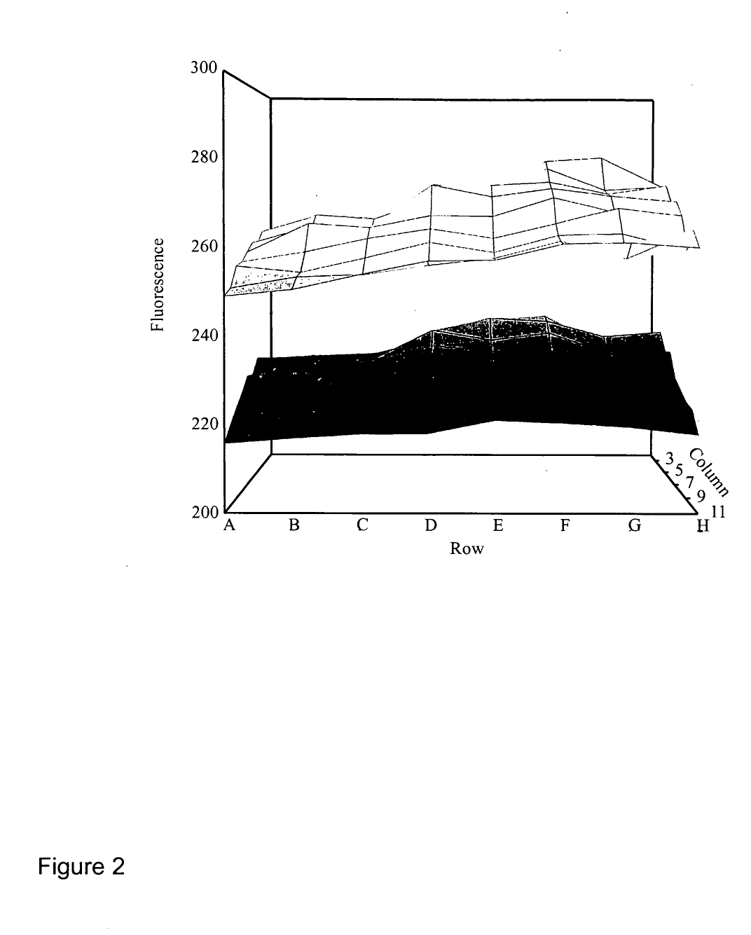

To determine the effectiveness of this assay, a blind experiment was performed in which 3 of the 96 wells were added to 5, 25, or 50 pmol DNA substrate that has the exact sequence of the fluorescent DNA reporter without the fluorescein label. This DNA would compete for RPA, therefore acting as an inhibitor. RPA was added to each of the wells followed by the addition of the competitive inhibitor and finally the fluorescein-labeled DNA. The results of the assay are presented in FIG. 2 and are plotted as percentage inhibition. Clearly, three wells were identified as having a significant inhibition of RPA binding to the fluorescein-labeled DNA. The effect of DMSO on reaction sensitivity and reproducibility was assessed and the results indicated no difference in assay sensitivity.

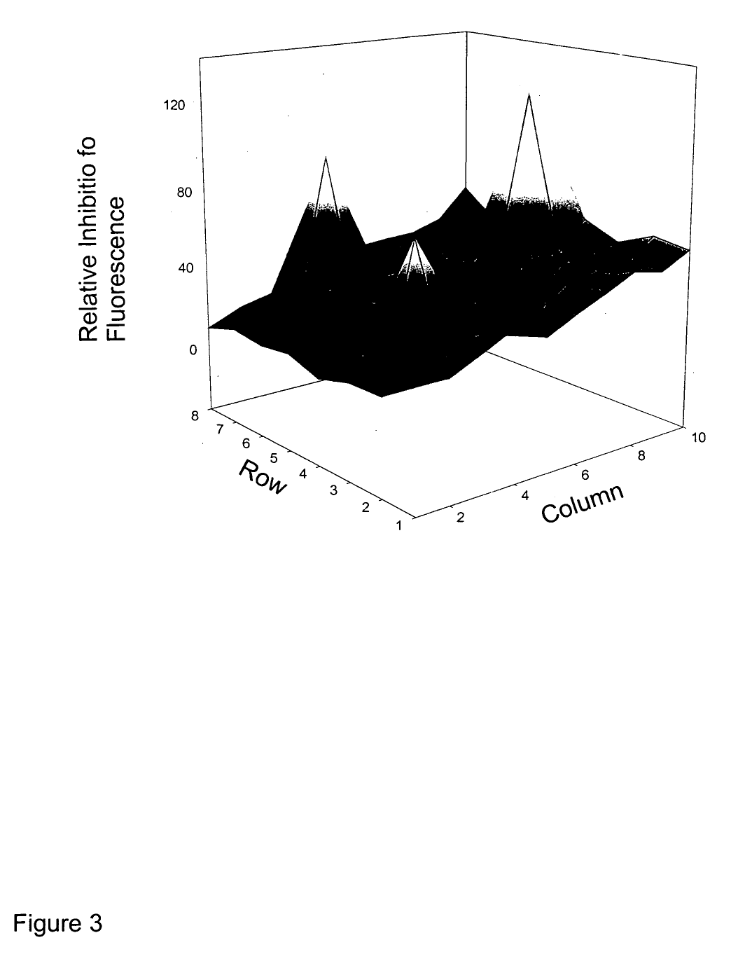

Based on these results, compounds from the NCI diversity set of pure and synthetic compounds were screened. The compounds received from the NCI were prepared in 100% DMSO. Sets of 80 compounds were screened on a 96-well plate with remaining wells being used for controls. Data from a representative plate are presented in FIG. 3. The results clearly demonstrate that there are a series of compounds capable of inhibiting RPA's DNA binding. It should be noted that color compounds were omitted from analysis regardless of their results because the color could interfere with the fluorescence reading and give incorrect results. In total, 2000 compounds were screened as described; of those screened, 79 were identified as inhibitors of RPA's DNA binding activity of RPA.

Analysis of Compound Binding via Fluorescence Polarization

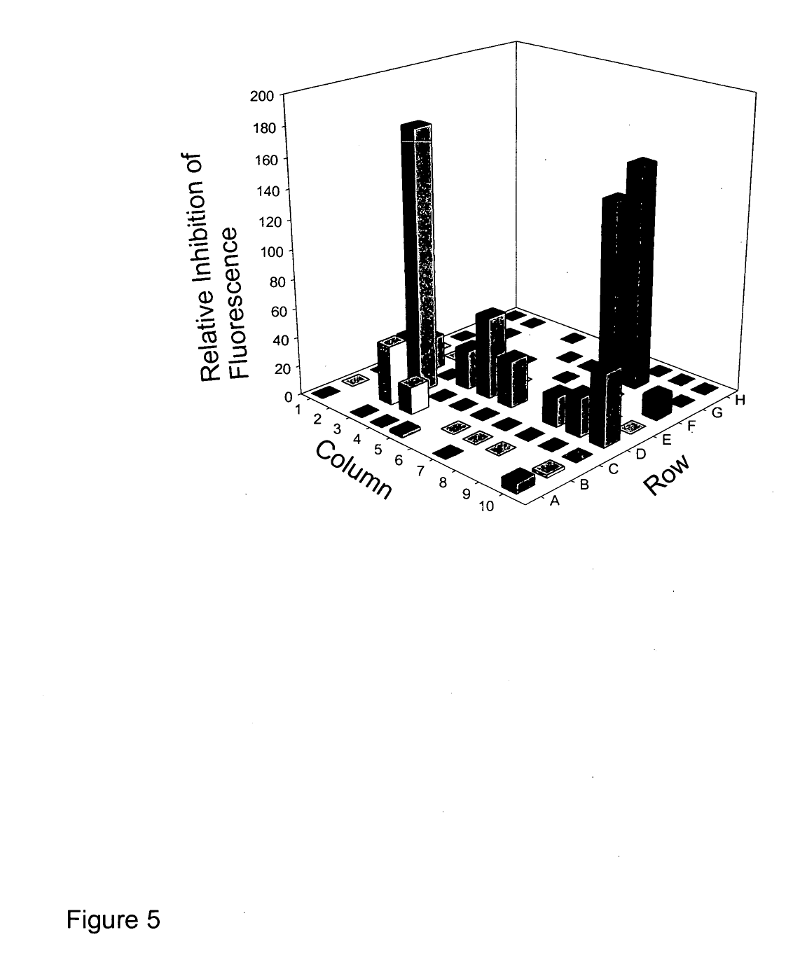

To more precisely determine if the method of inhibition was via an interaction with DNA or RPA, fluorescence polarization experiments were conducted. Fluorescence polarization is a measure of the rate at which an object tumbles or rotates in three-dimensional space. In this experiment, a fluorescein-labeled dT₃₀ substrate DNA is employed which results in a baseline polarization that is increased upon RPA binding; to the DNA and slowing its rotation. Using this method, it can be determined if the interaction of the compound was with DNA or RPA by constructing binding isotherms varying RPA at a fixed concentration of DNA with and without the addition of specific compounds. Using fluorescence polarization, compounds that exert their inhibitory effect through an interaction with the DNA were capable of being identified. Fluorescence polarization was performed by titrating RPA into a reaction containing fluorescein-labeled dT₃₀ DNA in the presence and absence of 20 μM compound (FIG. 5). The results presented in FIG. 5 demonstrate that in the control reactions performed in the absence of compounds, RPA binds the DNA as measured by the increase in r value with increasing RPA (FIG. 5, open circles). The analyses of RPA-DNA binding in reactions containing compound E51 yield significantly different results (filled circles). The analyses of RPA-DNA binding in reactions containing compound E51 yield significantly different results (filled circles). The maximum r value obtained in reactions containing compound E51 (r=0.13) was less than that observed for reactions performed without the addition of compound E51 (r=0.23), suggesting that the compound was decreasing the effective concentration of the DNA. This interpretation was supported by the fact that saturation was achieved at 8 pmol less RPA in the presence of compound E51 than that observed in the control experiment (15 pmol RPA). These data alone suggest that the compound was binding the DNA directly and slowing the rotation of the fluorescein-labeled DNA. This conclusion is further supported by experiments performed measuring the effect of compound 1:51 on polarization of the DNA in the absence of RPA. In these reactions, the r value of the DNA plus compound was slightly greater than the r value of the DNA without compound. In comparison, the analysis of compound D42 yielded results different from both the control and compound E51. Results with compound D42 (triangles) revealed a titration curve with a reduced initial slope, while the maximum r values were similar to the control reaction. The higher RPA concentration necessary to reach saturation suggests that the compound is interacting with RPA, therefore inhibiting the binding of RPA to the DNA substrate. In essence, the compound is removing the RPA from the reaction by binding it, resulting in the need for additional RPA to reach saturation.

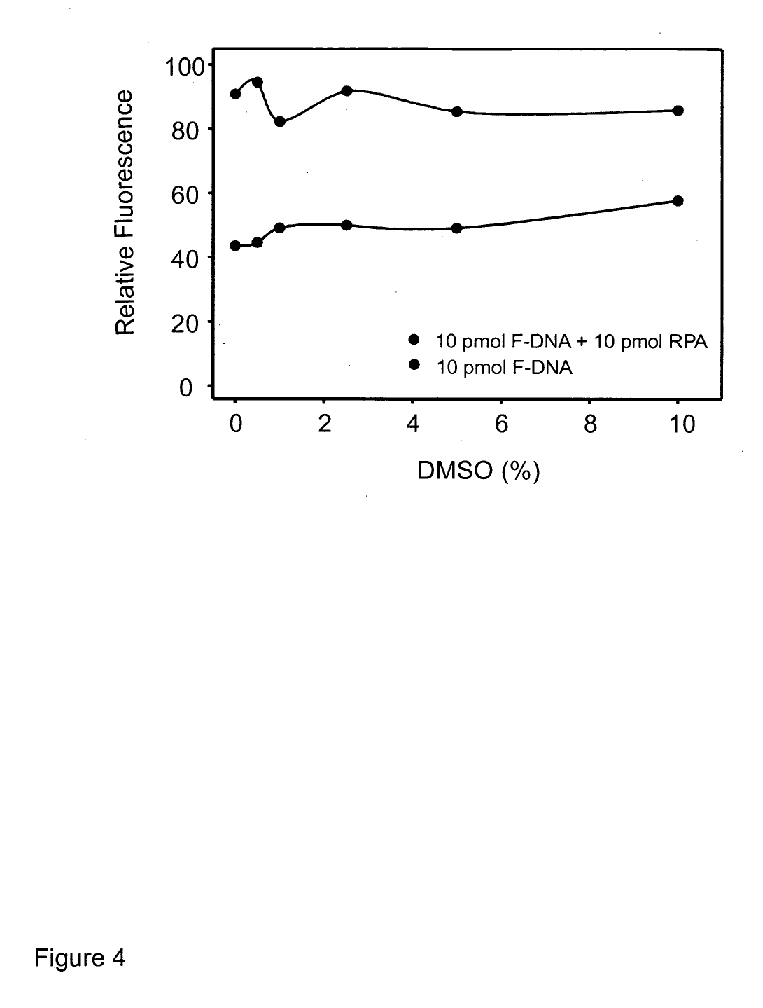

Libraries of small molecules are typically prepared in 100% dimethyl sulfoxide and thus any assay screening these libraries must be fairly robust and stable in reactions containing DMSO. The stability of the assay was assessed to various concentrations of DMSO and the results presented in FIG. 4 demonstrate that there s no significant difference in assay sensitivity or stability over a wide range of DMSO concentrations.

The final figure (FIG. 5) represents the screening data obtained from a single 96-well plate containing 80-small molecules obtained from the National Institutes of Health, National Cancer Institute Developmental Therapeutics Program diversity set. The results reveal that a number of compounds are capable of inhibiting RPAs DNA binding activity. Validation of these compounds has been performed and the results demonstrate that the assay allows the detection of small molecule inhibitors of RPA's DNA binding activity.

1. A homogenous high throughput assay for rapidly screening a plurality of compounds to determine degree of inhibition of Replication Protein A (RPA) binding of DNA by the compounds, the assay comprising:
a) adding a volume of RPA to a plurality of wells;
b) adding a volume of each of the plurality of compounds to each of the plurality of wells;
c) adding a volume of DNA labeled with a reporter to each of the plurality of wells, wherein the reporter emits a detectable signal;
d) measuring an amount of detectable signal emitted from each of the plurality of wells;
e) determining the degree of inhibition of RPA binding of DNA in each well by comparing the amount of detectable signal with an amount of background detectable signal wherein the background detectable signal is an amount determined by running the assay without step (b).

2. The assay as recited in claim 1 wherein the DNA is single stranded DNA.

3. The assay as recited in claim 1 further comprising a secondary screening step to identify the mechanism of inhibition, the secondary screening step comprising fluorescence polymerization.

4. The assay as recited in claim 1 wherein the reporter is a fluorescent reporter and the signal is fluorescence emission.

5. The assay as recited in claim 1 wherein the reporter comprises 5′ purine-bound fluorescein.

6. The assay as recited in claim 1 wherein the single stranded DNA comprises 5′ purine-bound fluorescein which shows an increase in fluorescence that is directly proportional to its binding with RPA.

7. The assay as recited in claim 2 wherein the single stranded DNA comprises SEQ ID NO: 1.

8. The assay as recited in claim 1 wherein the plurality of compounds is a library of compounds.

9. A fluorescently labeled single stranded DNA probe consisting essentially of SEQ ID NO: 1 bound with a fluorescent label.

10. A homogenous assay for screening a library of compounds for inhibition of Replication Protein A (RPA) binding of DNA, the assay comprising:
a) adding a liquid suspension of RPA to a plurality of wells;
b) adding a library of compounds to be screened for the inhibition to the plurality of wells;
c) adding a fluorescently labeled single-stranded DNA probe to the plurality of wells;
d) measuring an amount of fluorescence associated with each well; and
e) determining the degree of the inhibition by one or more of the compounds.

11. The assay as recited in claim 10 wherein the fluorescently labeled single-stranded DNA probe comprises 5′ purine-bound fluorescein.

12. The assay as recited in claim 10 wherein the single stranded DNA comprises SEQ ID NO: 1.

13. A method of treating a condition, disease or disorder by modulating DNA replication comprising:
a) screening compounds according to the assay recited in claim 1;
b) selecting those compounds which show the greatest degree of RPA inhibition;
c) using fluorescence polymerization to exclude those compounds which inhibit RPA via binding with the DNA;
d) formulating a pharmacological composition comprising one or more of any remaining compounds; and
e) administering the pharmacological composition to a subject in need of such treatment.

14. The method as recited in claim 13 wherein the condition, disease or disorder is cancer.

15. The method as recited in claim 13 wherein the condition, disease or disorder is acquired resistance to chemotherapeutic agents.

16. The method as recited in claim 13 wherein the condition, disease or disorder is a platinum-resistant carcinoma.

17. The method as recited in claim 13 wherein the condition, disease or disorder is ovarian cancer.

18. A method of treating a condition, disease or disorder benefited by the induction of cell apoptosis comprising:
a) screening compounds according to the assay recited in claim 1;
b) selecting those compounds which show the greatest degree of RPA inhibition;
c) using fluorescence polymerization to exclude those compounds which inhibit RPA via binding with the DNA;
d) formulating a pharmacological composition comprising one or more of any remaining compounds; and
e) administering the pharmacological composition to a subject in need of such treatment.

19. The method as recited in claim 18 wherein the condition, disease or disorder is cancer.

20. The method as recited in claim 18 wherein the condition, disease or disorder is acquired resistance to chemotherapeutic agents.

21. The method as recited in claim 18 wherein the condition, disease or disorder is a platinum-resistant carcinoma.

22. The method as recited in claim 18 wherein the condition, disease or disorder is a ovarian cancer.

23. A method for inhibiting DNA repair mechanisms along the Nucleotide Excision Repair (NER) pathway comprising: screening compounds according to the assay recited in claim 1; selecting those compounds which show the greatest degree of RPA inhibition; using fluorescence polymerization to exclude those compounds which inhibit RPA via binding with the DNA; formulating a pharmacological composition comprising one or more of any remaining compounds; and administering the pharmacological composition to a subject in need of such treatment.

24. A method of treating a condition, disease or disorder by modulation of the NER pathway comprising:
a) screening compounds according to the assay recited in claim 1;
b) selecting those compounds which show the greatest degree of RPA inhibition;
c) using fluorescence polymerization to exclude those compounds which inhibit RPA via binding with the DNA;
d) formulating a suitable pharmacological composition comprising one or more of any remaining compounds; and
e) administering the suitable pharmacological composition to a subject in need of such treatment.

25. The method as recited in claim 24 wherein the condition, disease or disorder is cancer.

26. The method as recited in claim 24 wherein the condition, disease or disorder is acquired resistance to chemotherapeutic agents.

27. The method as recited in claim 24 wherein the condition, disease or disorder is a platinum-resistant carcinoma.

28. The method as recited in claim 24 wherein the condition, disease or disorder is ovarian cancer.

29. A screen for a potential therapeutic agent for the enhancement of cancer chemotherapy wherein the enhancement comprises reversal of acquired resistance to DNA damaging agents, the screen comprising the assay as recited in claim 1.

30. The assay as recited in claim 1 wherein at least one of the steps is performed robotically.
